(19) United States
(12) Patent Application Publication
(10) Pub. No.: US 2010/0331861 A1
(43) Pub. Date: Dec. 30, 2010

(54) COSMETIC DEVICE

(75) Inventors: Kenichi Muraki, Hikone (JP); Kazuyuki Ohuchi, Hikone (JP)

Correspondence Address: WOLF GREENFIELD & SACKS, P.C., 600 ATLANTIC AVENUE, BOSTON, MA, 02210-2206, US

(73) Assignee: Panasonic Electric Works Co., LTD., Osaka (JP)

(21) Appl. No.: 12/820,231

(22) Filed: Jun. 22, 2010

(30) Foreign Application Priority Data
Jun. 25, 2009 (JP) 2009-151326

(51) Int. Cl. A61B 17/50

(52) U.S. Cl. 606/133

(57) ABSTRACT

A cosmetic device includes a grip, a keratin removal head detachably attached to the grip, and an epilator head detachably attached to the grip. Either one of the keratin removal head and epilator head is attached to the grip.

### CROSS-REFERENCE TO RELATED APPLICATIONS

This application is based upon and claims the benefit of priority from prior Japanese Patent Application No. 2009-151326, filed on Jun. 25, 2009, the entire contents of which are incorporated herein by reference.

### BACKGROUND OF THE INVENTION

The present invention relates to a cosmetic device.

Japanese Laid-Open Patent Publication No. 4-30818 describes a prior art example of a cosmetic device for removing skin keratin. The cosmetic device includes a head, which includes a rotatable scrubber, and a grip, which is formed integrally with the head. The scrubber is rotated and pressed against the skin to remove keratin.

### SUMMARY OF THE INVENTION

However, when removing keratin from a portion including body hair with the cosmetic device of the prior art, the body hair interferes with the keratin removal. In such a case, it becomes difficult to remove keratin from the skin.

The present invention provides a cosmetic device that facilitates the removal of skin keratin.

One aspect of the present invention is a cosmetic device for removing skin keratin and body hair. The cosmetic device includes a grip used for grasping, a keratin removal unit which removes skin keratin, and a hair removal unit which removes body hair. A selected one of or both of the keratin removal unit and the hair removal unit are attachable to the grip in a detachable manner.

Other aspects and advantages of the present invention will become apparent from the following description, taken in conjunction with the accompanying drawings, illustrating by way of example the principles of the invention.

### BRIEF DESCRIPTION OF THE DRAWINGS

The invention, together with objects and advantages thereof, may best be understood by reference to the following description of the presently preferred embodiments together with the accompanying drawings in which:

FIGS. 1(a) and 1(b) are respectively a front view and a side view showing a cosmetic device according to a first embodiment of the present invention;

FIG. 2 is a cross-sectional view of the cosmetic device taken along line 2-2 in FIG. 1(b);

FIG. 3 is a cross-sectional view of the cosmetic device shown in FIG. 1(b);

FIG. 4 is a from view showing a grip, a keratin removal head, and an epilator head;

FIGS. 5(a) and 5(b) are cross-sectional views showing the epilator head and respectively taken along line 5a-5a in FIGS. 4 and 5b-5b in FIG. 5;

FIG. 6 is a front view showing a cosmetic device according to a second embodiment of the present invention; and

FIGS. 7 and 8 are cross-sectional views showing the cosmetic device of FIG. 6.

### DETAILED DESCRIPTION OF THE PREFERRED EMBODIMENTS

A cosmetic device according to a first embodiment of the present invention will now be discussed with reference to the drawings.

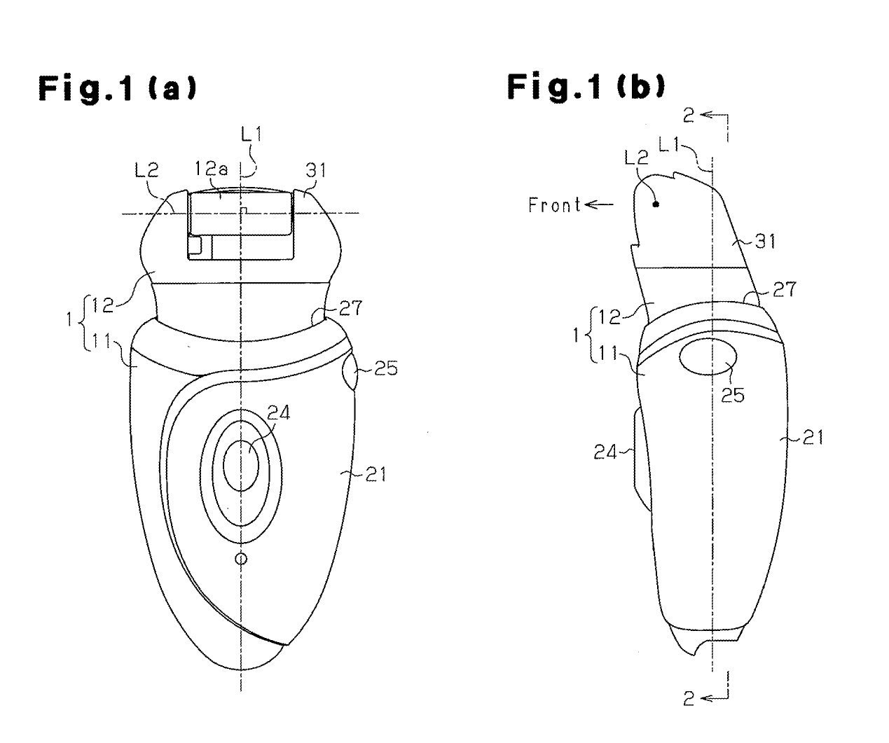

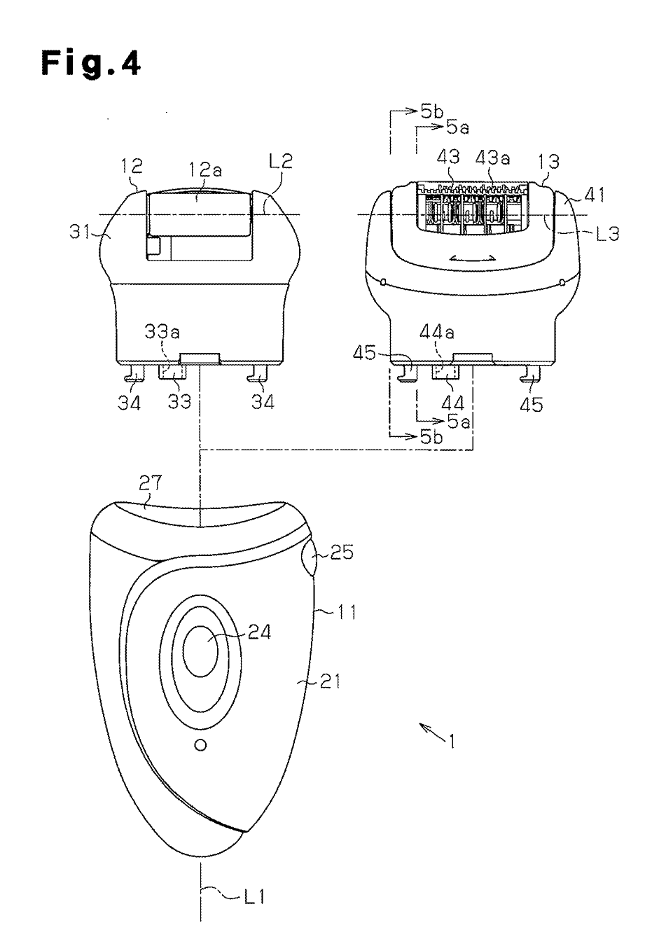

FIGS. 1(a) and 1(b) show a cosmetic device 1, which includes a grip 11, and a keratin removal head 12, which is used to remove keratin and attached to an upper part of the grip 11. The grip 11 is formed so as to allow a user to grasp it. The keratin removal head 12 includes a rotary cylinder 12a, which is rotatably supported and used to remove keratin. The rotary cylinder 12a is rotated and pressed against the skin to remove keratin from the skin. The cosmetic device 1 of the present embodiment allows for the keratin removal head 12 to be exchanged with an epilator head 13 (refer to FIG. 4). When a user removes keratin from a portion of the skin including body hair (treated portion) where keratin removal is difficult, the user first uses the epilator head 13 to remove hair from the skin portion. Then, the user exchanges the epilator head 13 with the keratin removal head 12 to remove keratin from the skin portion. The epilator head 13 is also referred to as a hair removal head.

The structure of the cosmetic device 1 will now be discussed in detail.

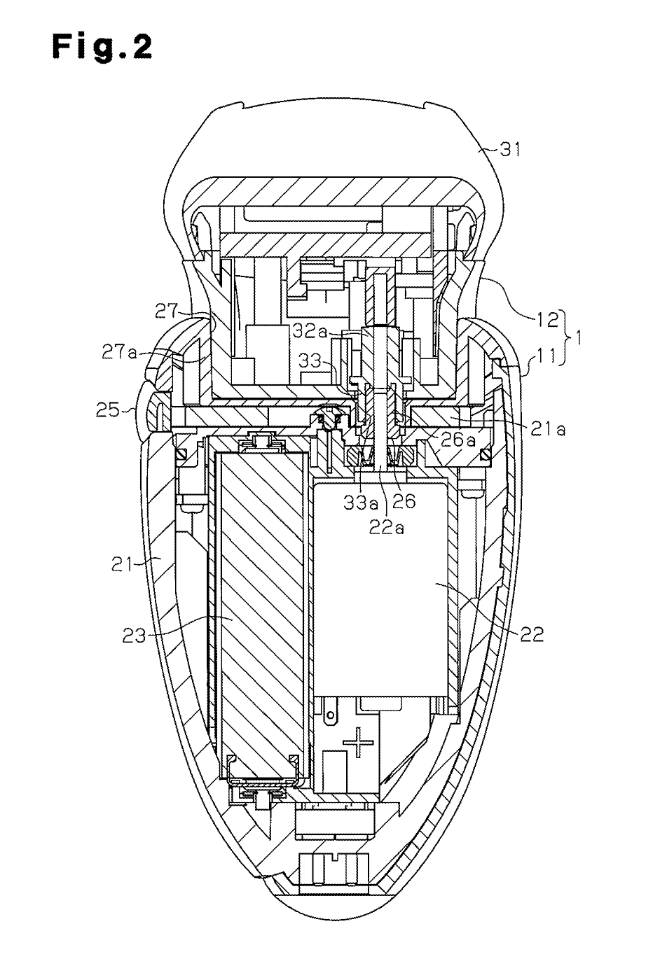

As shown in FIGS. 1 to 3, the grip 11 includes a housing 21 accommodating a motor 22, which serves as a drive source, and a rechargeable battery 23. A power switch 24 and a release button 25 are arranged on the housing 21. The power switch 24 is used to drive the motor 22. The release button 25 releases the keratin removal head 12 or epilator head 13 from the grip 11. The housing 21 of the grip 11 may have a water resistant structure that prevents water or the like from entering the grip 11.

The motor 22 has an output shaft 22a, which is fixed to an output member 26. In the illustrated example, the output member 26 includes a hexagonal fitting bushing 26a, which projects out of the housing 21 from an upper plate 21a.

The upper part of the grip 11 includes a head seat 27 (refer to FIG. 4). The head seat 27 may be a recess defined by the upper plate 21a and a side wall 27a. A basal part of either one of the keratin removal head 12 and the epilator head 13 is fitted to the head seat 27. This detachably attaches either one of the keratin removal head 12 and the epilator head 13 to the grip 11. The head seat 27 may also be referred to as a common seat or single seat.

The keratin removal head 12 includes a head housing 31, a reduction gear unit 32, and the rotary cylinder 12a. The reduction gear unit 32 is arranged in the head housing 31. The rotary cylinder 12a, which is used for keratin removal, is cylindrical and rotatably arranged in an upper part (distal part) of the head housing 31.

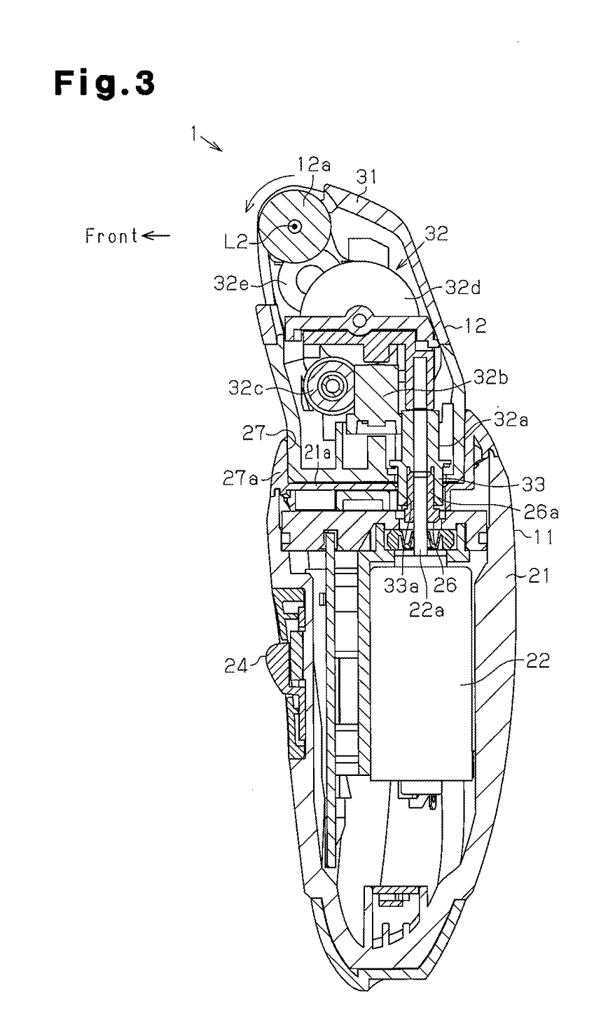

Referring to FIG. 3, the reduction gear unit 32 includes first to fifth gears 32a to 32e. The first gear 32a has a coupler 33, which projects from a basal part of the head housing 31 parallel to the longitudinal axis L1 of the cosmetic device 1 (grip 11). The coupler 33 is in conformance with the output member 26 of the grip 11. In the illustrated example, the coupler 33 includes a hexagonal fitting sleeve 33a. When the keratin removal head 12 is attached to the grip 11, the fitting sleeve 33a of the coupler 33 is fitted to the fitting bushing 26a of the output member 26. This conveys the rotation produced by the motor 22 to the first gear 32a. In this manner, the first gear 32a serves as a power input end of the keratin removal head 12. The rotation of the first gear 32a is slowed by the second gear 32b, third gear 32c, fourth gear 32d, and fifth gear 32e and consequently conveyed to the rotary cylinder 12a.

The rotary cylinder 12a has a rotation axis L2, which is orthogonal to the longitudinal axis L1 of the grip 11 when viewing the cosmetic device 1 from the front (the rotation axis L2 extends in a direction perpendicular to the longitudinal axis L1). The motor 22 drives and rotates the rotary cylinder 12a in the counterclockwise direction as viewed in FIG. 3. The rotary cylinder 12a has a circumferential surface to which scrubbing grains (scrubber) are arranged. When the motor 22 drives and rotates the rotary cylinder 12a, this produces friction between the rotary cylinder 12a and the skin and thereby removes keratin from the skin.

The rotary cylinder 12a is exposed from upper and front sides of the head housing 31. This allows for the rotary cylinder 12a removing keratin from not only the upper side but also from the front side. When removing keratin with the front exposed part of the rotary cylinder 12a, the grip 11 is held in a state inclined to the skin surface from which keratin is to be removed. Thus, the frictional force produced between the rotary cylinder 12a and the skin is communicated to the user who is holding the grip 11. This allows for stable keratin removal. Further, the rotation axis L2 of the rotary cylinder 12a is located frontward from the longitudinal axis L1 of the grip 11. Thus, when the grip 11 is held by the user, the front side of the rotary cylinder 12a is easily pressed against the treated portion of the skin.

As shown in FIG. 4, two hooks 34 project from the basal part of the keratin removal head 12. The grip 11 includes a fastener (not shown), which moves integrally with or in cooperation with the release button 25. When the keratin removal head 12 is attached to the grip 11, the fastener is engaged with the hooks 34 of the keratin removal head 12. This prevents separation of the keratin removal head 12 from the head seat 27. When the release button 25 is pushed, the fastener is moved together with the release button 25 so as to release the hooks 34 from the fastener. In this state, the keratin removal head 12 is detachable from the grip 11. The keratin removal head 12 is modularized. In other words, the keratin removal head 12 is a self-contained module that has the reduction gear unit 32 and the rotary cylinder 12a arranged in the head housing 31. This allows for the keratin removal head 12 to be easily attached to and detached from the grip 11.

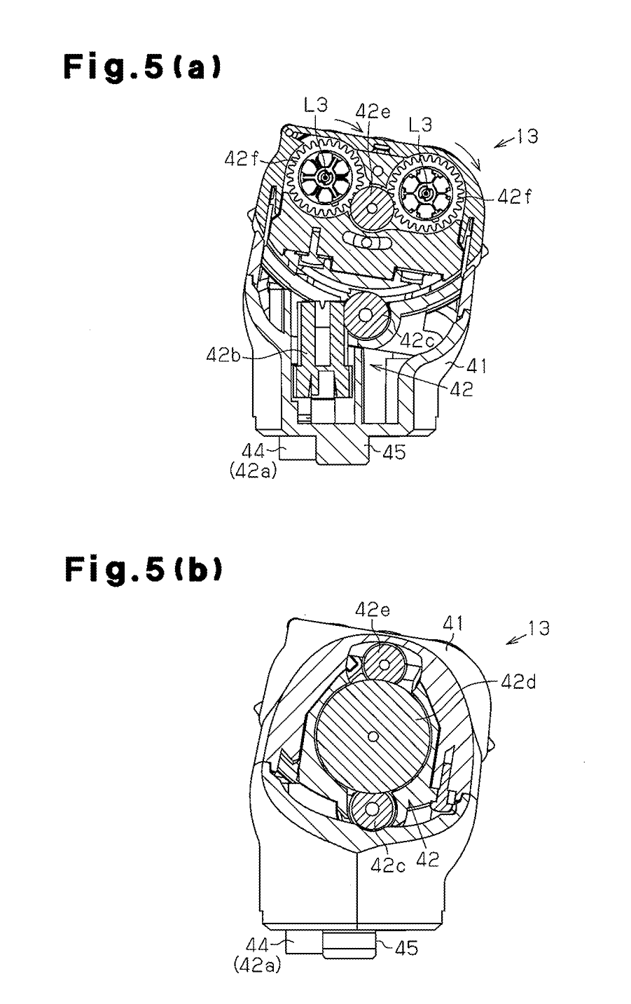

The epilator head 13, which is exchangeable with the keratin removal head 12, will now be described with reference to FIGS. 4 and 5(a) and 5(b). In the same manner as the basal part of the keratin removal head 12, the basal part of the epilator head 13 is formed to be attachable to the head seat 27 of the grip 11. Thus, the epilator head 13 is compatible with the keratin removal head 12. In the illustrated example, the basal part of the epilator head 13 is identical in shape and structure with the basal part of the keratin removal head 12.

The epilator head 13 includes a head housing 41, a reduction gear unit 42 (refer to FIGS. 5(a) and 5(b)), and two cylindrical epilator rotary cylinders 43 (removal rotary cylinder). The reduction gear unit 42 is accommodated in the head housing 41. The rotary cylinders 43 are rotatably arranged in an upper part (distal part) of the head housing 41.

The reduction gear unit 42 of the epilator head 13 includes first to sixth gears 42a to 42f. The first gear 32a has a coupler 44, which is shaped identically to the coupler 33 of the keratin removal head 12 and projects from a basal part of the head housing 41 parallel to the longitudinal axis L1 of the cosmetic device 1 (grip 11). When the basal part of the epilator head 13 is attached to the head seat 27, a fitting sleeve 44a of the coupler 44 is fitted to the output member 26 of the motor 22. This conveys the rotation produced by the motor 22 to the first gear 42a. In this manner, the first gear 42a serves as a power input end of the epilator head 13. The rotation of the first gear 42a is slowed by the second gear 42b, third gear 42c, fourth gear 42d, fifth gear 42e, and sixth gear 62f and consequently conveyed to each of the epilator rotary cylinders 43 (refer to FIGS. 5(a) and 5(b)).

Tweezers 43a, which serve as hair removal elements, are arranged on the circumferential surface of each epilator rotary cylinder 43. When the rotary cylinder 43 rotates, the tweezers 43a capture and pluck out hair from the skin surface. More specifically, the tweezers 43a each include a set of a fixed jaw and movable jaw. As the rotary cylinder 43 rotates, a lever extending through the rotary cylinder 43 moves along the rotation axis L3 of the rotary cylinder 43. This moves the movable jaw toward or away from the fixed jaw and thereby captures or releases hair. Such an epilator mechanism is known in the art and thus will not be described here in detail.

The reduction gear ratio of the reduction gear unit 42 in the epilator head 13 differs from the reduction gear ratio of the reduction gear unit 32 in the keratin removal head 12. The rotation speed of the keratin removal rotary cylinder 12a and the rotation speed of the epilator rotary cylinders 43 are set at values suitable for their functions. More specifically, the reduction gear ratio of the reduction gear unit 32 of the keratin removal head 12 is set to be greater than that of the reduction gear unit 42 of the epilator head 13. Further, the keratin removal rotary cylinder 12a is rotated with sufficient torque for removing keratin.

The keratin removal rotary cylinder 12a and the epilator rotary cylinders 43 have different diameters. More specifically, the diameter of the keratin removal rotary cylinder 12a is smaller than the diameter of the epilator rotary cylinders 43 so that the keratin removal rotary cylinder 12a is rotated with sufficient torque for removing keratin.

The rotation axis L3 of each epilator rotary cylinder 43 is orthogonal to the longitudinal axis L1 of the grip 11 when viewing the cosmetic device 1 from the front (the rotation axis L3 extends in a direction perpendicular to the longitudinal axis L1). The motor 22 drives and rotates each epilator rotary cylinder 43 in the clockwise direction as viewed in FIG. 5(a). In other words, in a state of use, the epilator rotary cylinders 43 rotate in the same direction as the keratin removal rotary cylinder 12a.

Two hooks 45, which are shaped identically to the hooks 34 of the keratin removal head 12, are formed on the basal part of the keratin removal head 12 and function in the same manner as the hooks 34 of the keratin removal head 12. More specifically, when the epilator head 13 is attached to the grip 11, the hooks 45 are engaged with the fastener of the grip 11. This prevents separation of the epilator head 13 from the head seat 27. When the release button 25 is pushed, the fastener is moved together with the release button 25 so as to release the hooks 45 from the fastener. In this state, the epilator head 13 is detachable from the grip 11. Further, in the same manner as the keratin removal head 12, the epilator head 13 is modularized. This allows for the epilator head 13 to be easily attached to and detached from the grip 11.

As described above, with the cosmetic device 1, when removing keratin from a portion including hair, the epilator head 13 is first attached to the head seat 27 to remove body hair from the skin. Then, the epilator head 13 is exchanged with the keratin removal head 12 to remove keratin. The cosmetic device 1 removes body hair with the epilator head 13 prior to keratin removal. This facilitates the removal of keratin.

The first embodiment has the advantages described below.

(1) In the first embodiment, the cosmetic device 1 includes the grip 11, which is held by the user, the keratin removal head 12, which is used to remove skin keratin, and the epilator head 13, which is used to remove body hair. The keratin removal head 12 and the epilator head 13 are selectively attached to the grip 11 in a detachable manner. This allows for the removal of body hair from the skin by attaching the epilator head 13 to the grip 11 and then the removal of keratin by exchanging the epilator head 13 with the keratin removal head 12. Thus, when removing keratin from a portion including body hair, the use of the cosmetic device 1 facilitates keratin removal without interference of body hair.

(2) In the first embodiment, the keratin removal head 12, which serves as a keratin removal unit, and the epilator head 13, which serves as a hair removal unit, are separate modules or discrete components. Further, the keratin removal head 12 and the epilator head 13 are each formed to be attachable to the grip 11 in a detachable manner. This facilitates the exchanging of the keratin removal head 12 and epilator head 13.

(3) In the first embodiment, the keratin removal head 12 includes the keratin removal rotary cylinder 12a, which has keratin removal scrubbing grains (scrubber) arranged on its circumferential surface. Further, the epilator head 13 includes the epilator rotary cylinders 43, each having the epilator tweezers 43a on its circumferential surface. This facilitates removal of hair and keratin.

(4) In the first embodiment, the rotary cylinders 12a and 43 have parallel rotation axes L2 and L3 (extending in the same direction in an attached state). Further, the rotation axes L2 and L3 are each orthogonal to the longitudinal axis L1 of the grip 11 when viewed from the front. This allows the user who is holding the grip 11 to easily press each of the rotary cylinders 12a and 43 against the treated portion.

(5) In the first embodiment, the rotary cylinders 12a and 43 are rotated in the same direction in a state of use. Thus, the direction of the force communicated to the user by the friction produced when pressing the rotating rotary cylinders 12a and 43 against the skin is the same for each of the rotary cylinders 12a and 43. As a result, the keratin removal head 12 and the epilator head 13 may be used with the same feel.

(6) In the first embodiment, the keratin removal rotary cylinder 12a is exposed from the upper and front sides of the head housing 31. This allows for the use of not only the upper side but also the front side of the rotary cylinder 12a and thereby facilitates the pressing of the keratin removal rotary cylinder 12a against the treated portion.

(7) In the first embodiment, the rotation axis L2 of the keratin removal rotary cylinder 12a is located frontward from the longitudinal axis L1 of the grip 11. Thus, when the grip 11 is held by the user, the front side of the keratin removal rotary cylinder 12a is easily pressed against the treated portion.

(8) In the first embodiment, the rotary cylinders 12a and 43 are each rotated by power from the same motor 22, which is arranged in the grip 11. Thus, there is no need for separate motors to be installed in the keratin removal head 12 and the epilator head 13. This avoids enlargement of the cosmetic device 1 and keeps the number of components low.

(9) In the first embodiment, the reduction gear ratio of the reduction gear unit 32 in the keratin removal head 12 differs from the reduction gear ratio of the reduction gear unit 42 in the epilator head 13. This allows for the rotation speed of the keratin removal rotary cylinder 12a and the rotation speed of the epilator rotary cylinders 43 to be set at values suitable for their functions. As a result, a high keratin scrubbing capability is obtained while also obtaining a high hair removal capability.

(10) In the first embodiment, the keratin removal rotary cylinder 12a and the epilator rotary cylinders 43 have different diameters. This allows for the diameter of the keratin removal rotary cylinder 12a and the diameter of the epilator rotary cylinders 43 to be set at values suitable for their functions. As a result, a high keratin scrubbing capability is obtained while also obtaining a high hair removal capability.

A cosmetic device according to a second embodiment of the present invention will now be discussed.

In the second embodiment, a cosmetic device 50 includes a grip 51. A keratin removal head 52 and an epilator head 53 are both incorporated in the grip 51. To avoid redundancy, like or same reference numerals are given to those components that are the same as the corresponding components of the first embodiment. The description centers on parts differing from the first embodiment.

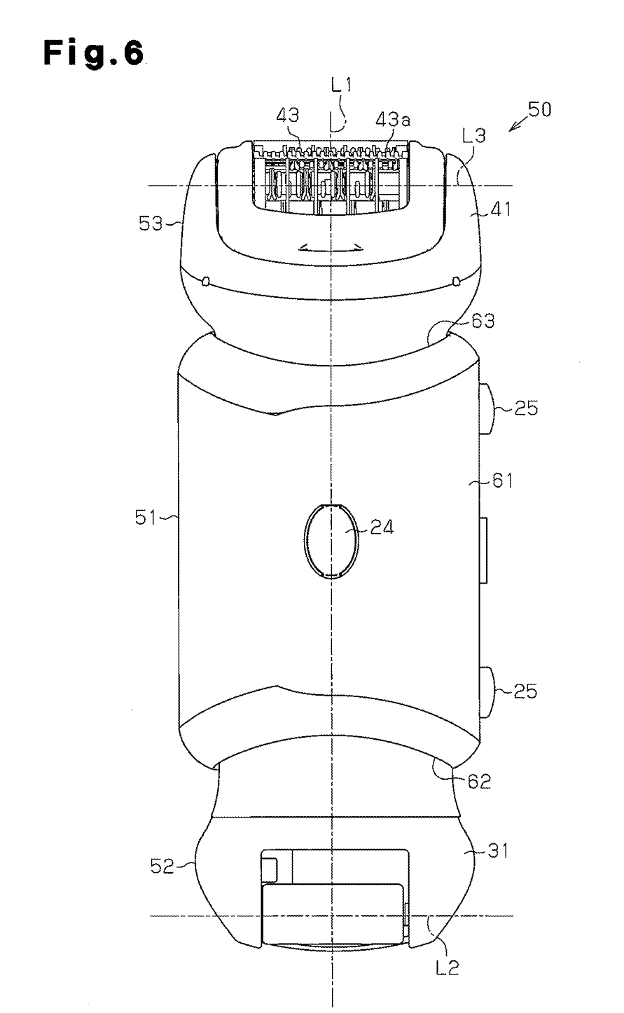

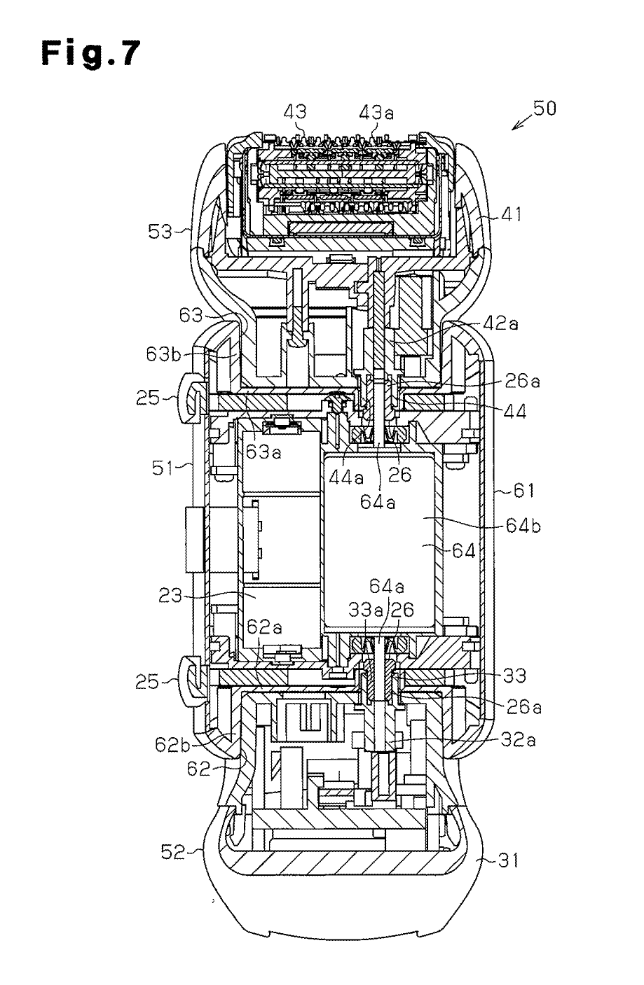

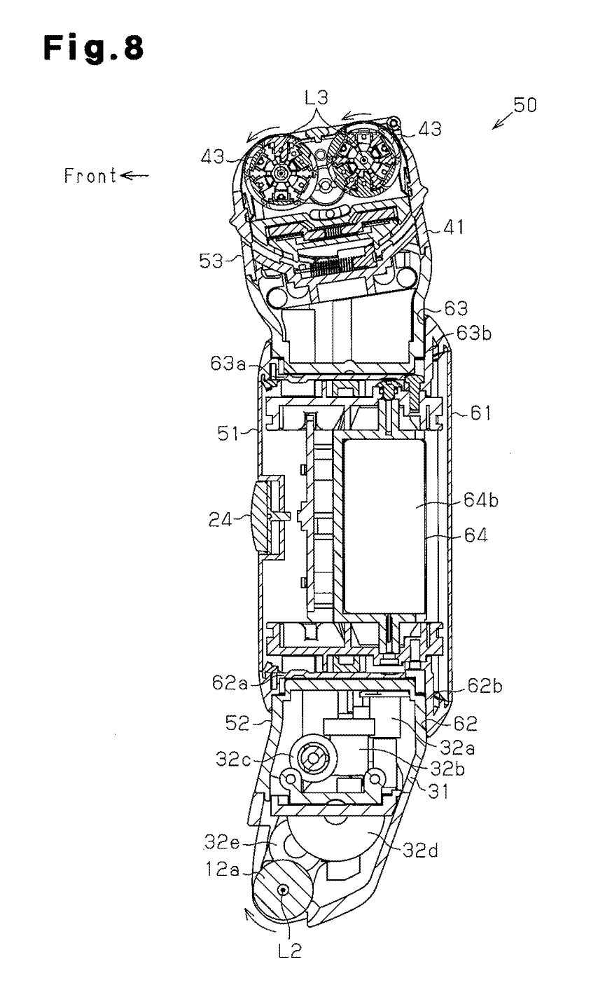

Referring to FIGS. 6 to 8, the grip 51 includes a housing 61, which has a longitudinal axis L1. The housing 61 has two axial ends defining a first head seat 62 and a second head seat 63 for attachment of a keratin removal head 52 and an epilator head 53, respectively. The head seats 62 and 63 respectively include bottoms 62a and 63a and side walls 62b and 63b. Further, the head seats 62 and 63 are formed to be hollow and extend toward the axially middle part of the housing 61. The head seats 62 and 63 of the second embodiment each have a structure that is similar to the head seat 27 of the first embodiment.

The housing 61 accommodates a motor 64, which serves as a motor 64, and a rechargeable battery 23. A power switch 24 and release buttons 25 are arranged on the grip 51. The power switch 24 is used to drive the motor 64. The release buttons 25 release the keratin removal head 52 and epilator head 53 from the corresponding head seats 62 and 63. The housing 61 of the grip 51 has a water resistant structure that prevents water or the like from entering the grip 51.

The motor 64 is of a two-output type and includes an output shaft 64a, the two ends of which project out of a motor housing 64b. The two ends of the output shaft 64a of the motor 64 respectively extend to the first and second head seats 62 and 63. Further, an output member 26, which is similar to that of the first embodiment, is fixed to each of the two ends. Each output member 26 includes a fitting bushing 26a, which projects out of the housing 61 from the bottom 62a or 63a of the corresponding head seat 62 or 63.

The grip 51 includes a fastener (similar to that of the first embodiment), which moves integrally with or in cooperation with a corresponding one of the release buttons 25. When the heads 52 and 53 are attached to the grip 51, the fastener is engaged with the hooks 34 or 45 of the corresponding keratin removal head 52 or epilator head 53. When the corresponding release button 25 is pushed, the fastener is released from the hooks 34 or 45. In this state, the corresponding one of the heads 52 and 53 is detachable from the grip 11.

The fitting sleeve 33a on the first gear 32a of the keratin removal head 52 is fitted to the fitting bushing 26a of the output member 26 corresponding to the first head seat 62. The fitting sleeve 44a on the first gear 42a of the epilator head 53 is fitted to the fitting bushing 26a of the output member 26 corresponding to the second head seat 63. This conveys the rotation produced by the motor 64 to the first gears 32a and 42a of the heads 52 and 53 and consequently to the rotary cylinders 12a and 43 of the heads 52 and 53. Further, the rotation axes L2 and L3 of the rotary cylinders 12a and 43 are parallel to each other and orthogonal to the longitudinal axis L1 of the grip 51 when viewed from the front (the rotation axes L2 and L3 extend perpendicular to the longitudinal axis L1).

When removing keratin from a portion including body hair with the cosmetic device 50, the epilator head 53 is first used to remove body hair from the skin. Then, the keratin removal head 12 is used to remove keratin. This facilitates keratin removal with the keratin removal head 52 without interference from body hair. Further, in a state of use (when the rotary cylinders 12a and 43 are pressing against the skin), the keratin removal rotary cylinder 12a and the epilator rotary cylinders 43 rotate in the same direction. Thus, the direction of the force communicated to the user by the friction produced when pressing the rotating rotary cylinders 12a and 43 against the skin is the same for each of the rotary cylinders 12a and 43. As a result, the keratin removal head 52 and the epilator head 53 may be used with the same feel.

The cosmetic device 50 of the second embodiment differs from the first embodiment in that the keratin removal head 52 and the epilator head 53 are both included in the grip 51. Nevertheless, the second embodiment has the same advantages as the first embodiment.

It should be apparent to those skilled in the art that the present invention may be embodied in many other specific forms without departing from the scope of the invention. Particularly, it should be understood that the present invention may be embodied in the following forms.

In the second embodiment, the keratin removal head 52 and the epilator head 53 are each modularized and discrete from the grip 51. However, the housing 61 of the grip 51 may be formed integrally with the head housings 31 and 41 of the heads 52 and 53 so as to form the grip 51 integrally with the keratin removal head 52 and the epilator head 53.

In the second embodiment, the output shaft 64a may include a clutch or the like so that when one of the keratin removal rotary cylinder 12a and epilator rotary cylinders 43 is driven, the other one is not rotated.

Each of the above-described embodiments includes one keratin removal rotary cylinder 12a and two epilator rotary cylinders 43. However, the present invention is not limited in such a manner. There may be two or more keratin removal rotary cylinders 12a. Further, there may be only one epilator rotary cylinder 43 or two or more epilator rotary cylinders 43.

In each of the above-described embodiments, the rotation axes L2 and L3 of the keratin removal rotary cylinder 12a and the epilator rotary cylinders 43 are orthogonal to the longitudinal axis of the grip 51 when viewed from the front. However, the present invention is not limited in such a manner, and the rotation axes L2 and L3 may extend in any direction in accordance with the structure.

In each of the above-described embodiments, the epilator head is used as a hair removal head. Instead, a shaver head for shaving off body hair may be used as the hair removal head.

The present examples and embodiments are to be considered as illustrative and not restrictive, and the invention is not to be limited to the details given herein, but may be modified within the scope and equivalence of the appended claims.

What is claimed is:

1. A cosmetic device for removing skin keratin and body hair, the cosmetic device comprising:
a grip used for grasping;
a keratin removal unit that removes skin keratin;
a hair removal unit that removes body hair;
wherein a selected one of or both of the keratin removal unit and the hair removal unit are attachable to the grip in a detachable manner.

2. The cosmetic device according to claim 1, wherein the keratin removal unit and the hair removal unit are respectively a modularized keratin removal head and a modularized hair removal head, and the keratin removal head and the hair removal head each are detachably attached to the grip.

3. The cosmetic device according to claim 1, wherein the keratin removal unit includes a keratin removal rotary cylinder that has a circumferential surface on which an scrubber is arranged to remove keratin; and
the hair removal unit includes a hair removal rotary cylinder that has a circumferential surface on which hair removal elements are arranged to remove hair.

4. The cosmetic device according to claim 3, wherein the grip has a longitudinal axis, and the keratin removal rotary cylinder and the hair removal rotary cylinder each have a rotation axis;
wherein the rotation axis of the keratin removal rotary cylinder and the rotation axis of the hair removal rotary cylinder are parallel to each other, and the rotation axis of the keratin removal rotary cylinder and the rotation axis of the hair removal rotary cylinder are orthogonal to the longitudinal axis of the grip as viewed from the front of the cosmetic device.

5. The cosmetic device according to claim 4, wherein the keratin removal rotary cylinder and the hair removal rotary cylinder rotate in the same direction in a state of use.

6. The cosmetic device according to claim 5, wherein the keratin removal rotary cylinder is exposed from an upper side and front side of the cosmetic device.

7. The cosmetic device according to claim 6, wherein the rotation axis of the keratin removal rotary cylinder is located frontward from the longitudinal axis of the grip.

8. The cosmetic device according to claim 7, wherein the rotary cylinders are each rotated by power produced by the same drive source that is arranged in the grip.

9. The cosmetic device according to claim 8, wherein the keratin removal unit and the hair removal unit each include a reduction gear unit that slows rotation produced by a motor serving as the drive source and transmits the rotation to the corresponding rotary cylinder.

10. The cosmetic device according to claim 9, wherein the keratin removal rotary cylinder and the hair removal rotary cylinder have different diameters.

11. The cosmetic device according to claim 1, wherein the grip includes a single seat; and
the keratin removal unit and the hair removal unit each have a basal part conforming to the single seat so that the keratin removal unit and the hair removal unit are compatible with each other.

12. The cosmetic device according to claim 11, wherein:
the grip includes a single drive source and an output member connected to the single drive source; and
the basal part of each of the keratin removal unit and the hair removal unit includes a coupler that is in conformance with the output member; and
the coupler is coupled to the output member to drive with the single drive source the one of the keratin removal unit and the hair removal unit corresponding to the coupled coupler.

13. The cosmetic device according to claim 1, wherein:
the grip includes two seats, a single drive source, and two output members connected to the single drive source and respectively arranged in the two seats, the two output members having the mutually same structure;
the basal part of each of the keratin removal unit and the hair removal unit includes a coupler that is in conformance with each of the two output members; and
the couplers coupled to the output members to drive with the single drive source both of the keratin removal unit and the hair removal unit.
